(19) United States
(12) Patent Application Publication
(10) Pub. No.: US 2016/0120484 A1
(43) Pub. Date: May 5, 2016

(54) PULSE OXIMETERWITH VOICE BROADCAST FUNCTION

(71) Applicant: AUGUSTINE Y. LIEN, Taipei City (TW)

(72) Inventor: AUGUSTINE Y. LIEN, Taipei City (TW)

(21) Appl. No.: 14/531,072

(22) Filed: Nov. 3, 2014

(51) Int. Cl. A61B 5/00; A61B 5/1455

(52) U.S. Cl. CPC A61B 5/741; A61B 5/14552; A61B 5/7278; A61B 2562/0238

(57) ABSTRACT

A pulse oximeter with a voice broadcast function for voice-broadcasting pulse rate-related information and blood oxygen level-related information is introduced. The pulse oximeter includes a pulse blood oxygen sensor, a signal processing circuit, and a loudspeaker. The pulse blood oxygen sensor senses a user's pulse rate-related information and blood oxygen level-related information and sends a physiological signal carrying the pulse rate-related information and the blood oxygen level-related information. The signal processing circuit is electrically connected to the pulse blood oxygen sensor and adapted to receive and convert the physiological signal into the pulse rate-related information and the blood oxygen level-related information and send the pulse rate-related information and the blood oxygen level-related information. The loudspeaker is electrically connected to the signal processing circuit and adapted to receive and voice-broadcast the pulse rate-related information and the blood oxygen level-related information.

### FIELD OF THE INVENTION

The present invention relates to pulse oximeters, and more particularly, to a pulse oximeter with a voice broadcast function.

### BACKGROUND OF THE INVENTION

During surgery, anesthesiologists monitor patients' physiological functions to ensure that the surgery can be performed safely and smoothly on the patients. In this regards, the anesthesiologists use various medical devices to monitor the patients' vital signs, namely body temperature, blood pressure, pulse rate, respiration rate, and, if necessary, additional vital signs, including blood oxygen level. These measurements are displayed on the monitors of the medical devices.

A conventional medical device, such as a pulse oximeter, measures blood oxygen level and pulse rate, and the measurements are displayed on a monitor of the pulse oximeter. Normally, it is an anesthesiologist who reads the measurements displayed on the pulse oximeter and reports verbally and instantly to the surgeon whatever changes in the measurement. But, during an operation performed by a surgeon, the surgeon has to glimpse at the measurements displayed on the pulse oximeter at times in some situations, for example, a cost-cutting scenario, a makeshift scenario, and a poor communication scenario.

As the medical service market is increasingly competitive worldwide, hospitals around the world cut staffing costs by hiring less anesthesiologists than needed. As a result, due to a shortage of anesthesiologists, each working anesthesiologist has to take care of two or more operating rooms simultaneously. And, in case an operation is simple and short, no anesthesiologist will be assigned to the operating room at all.

When equipped with an operating room, makeshift hospitals in a battlefield, at the scene of a natural disaster, and on a ship at sea, for example, seldom have any anesthesiologist.

Verbal communication between a surgeon and an anesthesiologist in an operating room is never free of errors, which is especially evident to a surgery team of multiple nationalities. In this regard, foolproof automation of vital sign monitoring is a good alternative but is not always affordable.

Hence, it is advantageous for surgeons to dispense with the need to glimpse at the measurements displayed on the monitor at times, so as to focus on the ongoing surgery.

### SUMMARY OF THE INVENTION

In view of the aforesaid drawbacks of the prior art, it is an objective of the present invention to enable surgeons to dispense with the need to glimpse at the measurements displayed on monitors at times, so as to focus on the ongoing surgery.

In order to achieve the above and other objectives, the present invention provides a pulse oximeter with a voice broadcast function. The pulse oximeter comprises a pulse blood oxygen sensor, a signal processing circuit, and a loudspeaker. The pulse blood oxygen sensor is adapted for sensing a pulse rate-related information and a blood oxygen level-related information and sending a physiological signal carrying the pulse rate-related information and the blood oxygen level-related information. The signal processing circuit is electrically connected to the pulse blood oxygen sensor and adapted to receive the physiological signal, convert the physiological signal into the pulse rate-related information and the blood oxygen level-related information, and send the pulse rate-related information and the blood oxygen level-related information. The loudspeaker is electrically connected to the signal processing circuit and adapted to receive the pulse rate-related information and the blood oxygen level-related information and voice-broadcast the pulse rate-related information and the blood oxygen level-related information.

In an embodiment of the present invention, the pulse blood oxygen sensor comprises a light emitting unit, a light receiving unit, and an optical signal processing unit, wherein the light emitting unit and the light receiving unit are disposed at two opposite sides of a portion of a user's body, wherein the light emitting unit emits an incident light beam to penetrate the user's body and exit the user's body in form of an outgoing light beam, wherein the light receiving unit receives the outgoing light beam and sends a data indicative of light intensity of the outgoing light beam, wherein the optical signal processing unit is electrically connected to the light receiving unit and adapted to receive and convert the data indicative of the light intensity of the outgoing light beam into the physiological signal. The light emitting unit comprises a red light source and an infrared light source, the red light source is a red light-emitting diode, the infrared light source is an infrared light-emitting diode.

In another embodiment of the present invention, the pulse blood oxygen sensor comprises a light emitting unit, a light receiving unit, and an optical signal processing unit, wherein the light emitting unit and the light receiving unit are disposed on the same side of a portion of a user's body, wherein the light emitting unit emits an incident light beam, wherein the incident light beam falls on the user's body and then reflects off the user's body in form of a reflecting light beam, wherein the light receiving unit receives the reflecting light beam and sends a data indicative of light intensity of the reflecting light beam, wherein the optical signal processing unit is electrically connected to the light receiving unit and adapted to receive and convert the data indicative of the light intensity of the reflecting light beam into a physiological signal. The light emitting unit comprises a red light source and an infrared light source, the red light source is a red light-emitting diode, the infrared light source is an infrared light-emitting diode.

In yet another embodiment of the present invention, the pulse oximeter further comprises an audio receiver electrically connected to the signal processing circuit and adapted to generate a voice control signal in accordance with an external audio message received, wherein the voice control signal enables the signal processing circuit to enter an operating mode for receiving the physiological signal and sending the pulse rate-related information and the blood oxygen level-related information and enables the signal processing circuit to enter a low power consumption mode.

Accordingly, the present invention provides a pulse oximeter which enables surgeons to dispense with the need to glimpse at the measurements displayed on a monitor, so as to focus on the ongoing surgery.

### BRIEF DESCRIPTION OF THE DRAWINGS

Objectives, features, and advantages of the present invention are hereunder illustrated with specific embodiments in conjunction with the accompanying drawings, in which:

FIG. 1 is a schematic view of a pulse oximeter according to an embodiment of the present invention;

FIG. 2A is a schematic view of a pulse blood oxygen sensor of the pulse oximeter in the first aspect according to the embodiment of the present invention;

FIG. 2B is a schematic view of the pulse blood oxygen sensor of the pulse oximeter in the second aspect according to the embodiment of the present invention; and

FIG. 3 is a schematic view of another pulse oximeter according to another embodiment of the present invention.

### DETAILED DESCRIPTION OF THE PREFERRED EMBODIMENTS

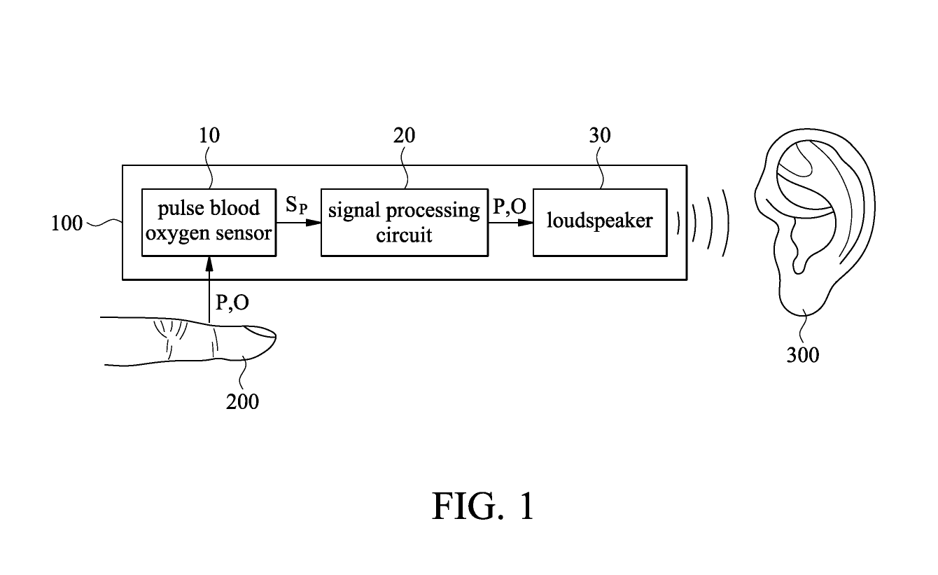

Referring to FIG. 1, there is shown a schematic view of a pulse oximeter 100 according to an embodiment of the present invention. As shown in the diagram, the pulse oximeter 100 comprises a pulse blood oxygen sensor 10, a signal processing circuit 20, and a loudspeaker 30.

The pulse blood oxygen sensor 10 is designed to come into contact with a portion of a user's body, such as the user's fingertip 200. The pulse blood oxygen sensor 10 senses a pulse rate-related information P and a blood oxygen level-related information O of the user and sends a physiological signal Sp carrying the pulse rate-related information P and the blood oxygen level-related information O. In this embodiment, the pulse blood oxygen sensor 10 senses the user's pulse rate-related information P and blood oxygen level-related information O by operating in a non-invasive manner, for an illustrative purpose. However, in a variant embodiment, the pulse blood oxygen sensor 10 senses the user's pulse rate-related information P and blood oxygen level-related information O by operating in an invasive manner so as to achieve enhanced accuracy as needed. The pulse blood oxygen sensor 10 is further described below to illustrate how it operates in two common non-invasive modes.

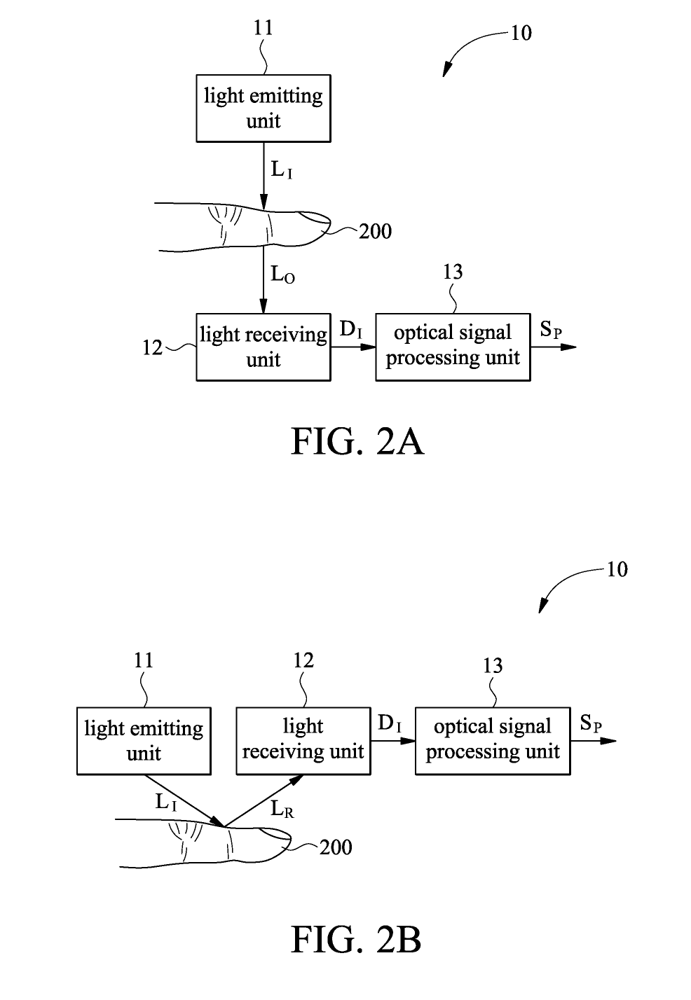

Referring to FIG. 2A, there is shown a schematic view of the pulse blood oxygen sensor 10 of the pulse oximeter in the first aspect according to the embodiment of the present invention. The pulse blood oxygen sensor 10 comprises a light emitting unit 11, a light receiving unit 12, and an optical signal processing unit 13. The light emitting unit 11 and the light receiving unit 12 are designed to come into contact with the opposite sides of a portion of the user's body, such as the fingertip 200. The optical signal processing unit 13 is electrically connected to the light receiving unit 12. The light emitting unit 11 emits an incident light beam LI which falls on the fingertip 200. In practice, the incident light beam LI is an electromagnetic wave which can penetrate the fingertip 200. After penetrating the fingertip 200, the otherwise incident light beam LI turns into an outgoing light beam L_{O}. As its name suggests, the outgoing light beam L_{O} exits the fingertip 200. Afterward, the light receiving unit 12 receives the outgoing light beam L_{O}, converts the outgoing light beam L_{O} into a data D_{I} indicative of the light intensity of the outgoing light beam L_{O}, and sends the data D_{I} to the optical signal processing unit 13. After receiving the data D_{I}, the optical signal processing unit 13 converts the data D_{I} into the physiological signal Sp.

Referring to FIG. 2B, there is shown a schematic view of the pulse blood oxygen sensor 10 of the pulse oximeter in the second aspect according to the embodiment of the present invention. The first and second aspects of the embodiment of the present invention share some common features, that is, the pulse blood oxygen sensor 10 comprises the light emitting unit 11, the light receiving unit 12, and the optical signal processing unit 13, wherein the optical signal processing unit 13 is electrically connected to the light receiving unit 12. But the first and second aspects of the embodiment of the present invention are distinguished from each other by the features described below. In the second aspect according to the embodiment of the present invention, both the light emitting unit 11 and the light receiving unit 12 are positioned on the same side of the fingertip 200, such that the incident light beam LI emitted from the light emitting unit 11 falls on the fingertip 200 to turn into a reflecting light beam LR which reflects off the fingertip 200. The light receiving unit 12 receives the reflecting light beam LR and converts the reflecting light beam LR into a data D_{I} indicative of the light intensity of the reflecting light beam LR. The processing unit 13 receives the data D_{I} and converts the data D_{I} into the physiological signal Sp.

The amounts of the light emitting unit 11, the incident light beam LI, the outgoing light beam L_{O}, the reflecting light beam LR and the data D_{I} of the foregoing pulse blood oxygen sensor 10 are respectively one for an illustration. However, the light emitting unit 11 can comprise a red light source and an infrared light source. The red light source and the infrared light source emit a red light and an infrared light to the fingertip 200, respectively. The optical signal processing unit 13 receives a red light data and an infrared data transmitted or reflected from the fingertip 200 and converts the red light data and the infrared data into the physiological signal Sp. The red light source of the light emitting unit 11 can be a red light-emitting diode, the infrared light source of the light emitting unit 11 can be an infrared light-emitting diode.

There are rhythmic changes in the inner diameter of arterioles in the fingertip 200, because the amount of blood passing through the arterioles in the fingertip 200 changes rhythmically as the heart expands and contracts rhythmically. Both the data D_{I} indicative of the light intensity of the outgoing light beam L_{O} in the first aspect of the embodiment of the present invention and the data D_{I} indicative of the light intensity of the reflecting light beam LR in the second aspect of the embodiment of the present invention change with the volume of blood passing through the arterioles in the fingertip 200, and the changes are detected by the light receiving unit 12 in real time so as to create the user's pulse rate-related information.

Similarly, both the data D_{I} indicative of the light intensity of the outgoing light beam L_{O} in the first aspect of the embodiment of the present invention and the data D_{I} indicative of the light intensity of the reflecting light beam LR in the second aspect of the embodiment of the present invention change with the blood oxygen level, and the changes are detected by the light receiving unit 12 in real time so as to create the user's blood oxygen level-related information.

In practice, changes attributed to the data D_{I} and detected by the light receiving unit 12 are measured in terms of light absorbance. It is because light intensity sensed by the light receiving unit 12 is inversely proportional to light absorbance of a matter, such as blood, and depends on the volume of blood passing through blood vessels and blood oxygen level.

Accordingly, in the first and second aspects according to the embodiment of the present invention, it is practicable to sense the user's pulse rate-related information P and blood oxygen level-related information O with the pulse blood oxygen sensor 10 and send the physiological signal Sp indicative of the pulse rate-related information P and the blood oxygen level-related information O.

Referring to FIG. 1, the signal processing circuit 20 is electrically connected to the pulse blood oxygen sensor 10 and adapted to receive the physiological signal S_{P}, convert the physiological signal S_{P} into the pulse rate-related information P and the blood oxygen information O, and send the pulse rate-related information P and the blood oxygen level-related information O.

The loudspeaker 30 is electrically connected to the signal processing circuit 20 and adapted to receive the pulse rate-related information P and the blood oxygen level-related information O and then voice-broadcast the pulse rate-related information P and the blood oxygen level-related information O. In addition, the pulse oximeter 100 can also electrically connect with a physiological monitor and use a loudspeaker of the physiological monitor to voice-broadcast the pulse rate-related information P and the blood oxygen level-related information O.

In practice, immediately before surgery, the pulse oximeter 100 is mounted on the patient's body in a manner that the patient's fingertip is in contact with the pulse blood oxygen sensor 10, such that the light emitting unit 11, the light receiving unit 12, and the optical signal processing unit 13 of the pulse blood oxygen sensor 10 operate in conjunction with each other to generate the physiological signal Sp indicative of the pulse rate-related information P and the blood oxygen level-related information O. Afterward, the signal processing circuit 20 receives the physiological signal Sp, converts the physiological signal Sp into the pulse rate-related information P and the blood oxygen level-related information O, and sends the pulse rate-related information P and the blood oxygen level-related information O. At last, after receiving the pulse rate-related information P and the blood oxygen level-related information O, the loudspeaker 30 voice-broadcasts the pulse rate-related information P and the blood oxygen level-related information O. Hence, during the surgery, the surgeon is informed of the patient's pulse rate-related information P and blood oxygen level-related information O by listening (with the surgeon's ear 300) to the loudspeaker 30, such that surgeon can focus on the surgeon.

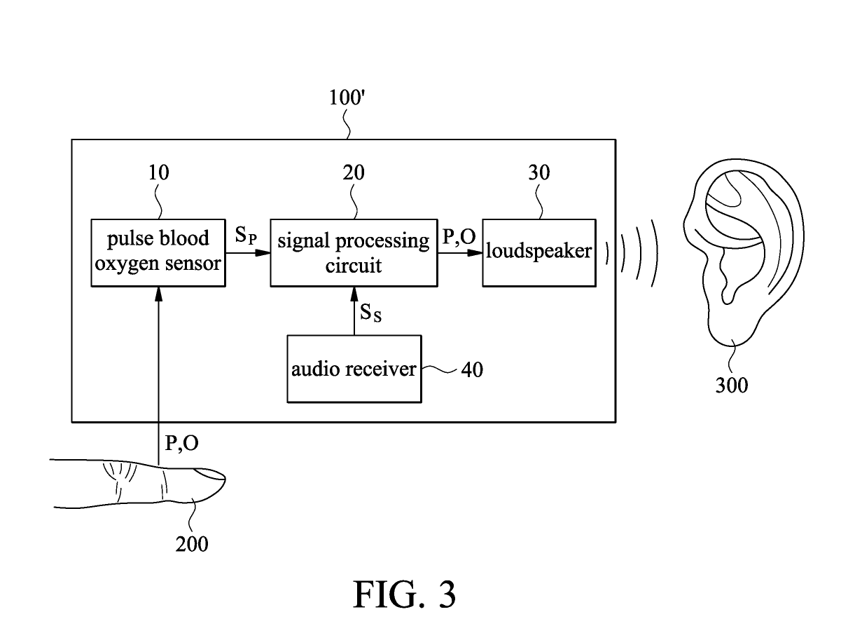

Referring to FIG. 3, there is shown a schematic view of a pulse oximeter 100′ according to another embodiment of the present invention. In addition to the pulse blood oxygen sensor 10, the signal processing circuit 20, and the loudspeaker 30,

the pulse oximeter 100′ further comprises an audio receiver 40. The audio receiver 40 functions like a microphone for perceiving ambient sound. The audio receiver 40 is electrically connected to the signal processing circuit 20 and adapted to be operated by a surgeon or a medical technician to adjust the pulse oximeter 100′ in a voice-controlled manner. The surgeon's or medical technician's voice is recorded on a storage component (not shown) of the signal processing circuit 20 in advance. Furthermore, the signal processing circuit 20 analyzes the ambient sound received by the audio receiver 40 and thus generates a control instruction accordingly. Also, the signal processing circuit 20 generates a voice control signal S_{S} in accordance with an external audio message given by the surgeon or medical technician. The voice control signal S_{S} enables the signal processing circuit 20 to enter an operating mode for receiving the physiological signal Sp and sending the pulse rate-related information P and the blood oxygen level-related information O. Also, the voice control signal S_{S} enables the signal processing circuit 20 to enter a low power consumption mode (also known as sleeping mode). Hence, the pulse oximeter 100′ wakens or enters a sleeping mode by a surgeon or medical technician and in a voice-controlled manner.

In conclusion, the pulse oximeter of the present invention is equipped with the pulse blood oxygen sensor, the signal processing circuit, and the loudspeaker whereby, during an operation being performed by a surgeon on a patient, the surgeon listens to the loudspeaker for pulse rate-related information and the blood oxygen level-related information pertaining to the patient, such that the surgeon can focus on the ongoing surgery.

The present invention is disclosed above by preferred embodiments. However, persons skilled in the art should understand that the preferred embodiments are illustrative of the present invention only, but should not be interpreted as restrictive of the scope of the present invention. Hence, all equivalent modifications and replacements made to the aforesaid embodiments should fall within the scope of the present invention. Accordingly, the legal protection for the present invention should be defined by the appended claims.

What is claimed is:

1. A pulse oximeter with a voice broadcast function, comprising:
a pulse blood oxygen sensor for sensing a pulse rate-related information and a blood oxygen level-related information and sending a physiological signal carrying the pulse rate-related information and the blood oxygen level-related information;
a signal processing circuit electrically connected to the pulse blood oxygen sensor and adapted to receive the physiological signal, convert the physiological signal into the pulse rate-related information and the blood oxygen level-related information, and send the pulse rate-related information and the blood oxygen level-related information; and
a loudspeaker electrically connected to the signal processing circuit and adapted to receive the pulse rate-related information and the blood oxygen level-related information and voice-broadcast the pulse rate-related information and the blood oxygen level-related information.

2. The pulse oximeter of claim 1, wherein the pulse blood oxygen sensor comprises a light emitting unit, a light receiving unit, and an optical signal processing unit, wherein the light emitting unit and the light receiving unit are disposed at two opposite sides of a portion of a user's body, wherein the light emitting unit emits an incident light beam to penetrate the user's body and exit the user's body in form of an outgoing light beam, wherein the light receiving unit receives the outgoing light beam and sends a data indicative of light intensity of the outgoing light beam, wherein the optical signal processing unit is electrically connected to the light receiving unit and adapted to receive and convert the data indicative of the light intensity of the outgoing light beam into the physiological signal.

3. The pulse oximeter of claim 2, wherein the light emitting unit comprises a red light source and an infrared light source, the red light source is a red light-emitting diode, the infrared light source is an infrared light-emitting diode.

4. The pulse oximeter of claim 1, wherein the pulse blood oxygen sensor comprises a light emitting unit, a light receiving unit, and an optical signal processing unit, wherein the light emitting unit and the light receiving unit are disposed on a same side of a portion of a user's body, wherein the light emitting unit emits an incident light beam, wherein the incident light beam falls on the user's body and then reflects off the user's body in form of a reflecting light beam, wherein the light receiving unit receives the reflecting light beam and sends a data indicative of light intensity of the reflecting light beam, wherein the optical signal processing unit is electrically connected to the light receiving unit and adapted to receive and convert the data indicative of the light intensity of the reflecting light beam into a physiological signal.

5. The pulse oximeter of claim 4, wherein the light emitting unit comprises a red light source and an infrared light source, the red light source is a red light-emitting diode, the infrared light source is an infrared light-emitting diode.

6. The pulse oximeter of claim 1, further comprising an audio receiver electrically connected to the signal processing circuit and adapted to generate a voice control signal in accordance with an external audio message received, wherein the voice control signal enables the signal processing circuit to enter an operating mode for receiving the physiological signal and sending the pulse rate-related information and the blood oxygen level-related information and enables the signal processing circuit to enter a low power consumption mode.

7. The pulse oximeter of claim 2, further comprising an audio receiver electrically connected to the signal processing circuit and adapted to generate a voice control signal in accordance with an external audio message received, wherein the voice control signal enables the signal processing circuit to enter an operating mode for receiving the physiological signal and sending the pulse rate-related information and the blood oxygen level-related information and enables the signal processing circuit to enter a low power consumption mode.

8. The pulse oximeter of claim 3, further comprising an audio receiver electrically connected to the signal processing circuit and adapted to generate a voice control signal in accordance with an external audio message received, wherein the voice control signal enables the signal processing circuit to enter an operating mode for receiving the physiological signal and sending the pulse rate-related information and the blood oxygen level-related information and enables the signal processing circuit to enter a low power consumption mode.

9. The pulse oximeter of claim 4, further comprising an audio receiver electrically connected to the signal processing circuit and adapted to generate a voice control signal in accordance with an external audio message received, wherein the voice control signal enables the signal processing circuit to enter an operating mode for receiving the physiological signal and sending the pulse rate-related information and the blood oxygen level-related information and enables the signal processing circuit to enter a low power consumption mode.

10. The pulse oximeter of claim 5, further comprising an audio receiver electrically connected to the signal processing circuit and adapted to generate a voice control signal in accordance with an external audio message received, wherein the voice control signal enables the signal processing circuit to enter an operating mode for receiving the physiological signal and sending the pulse rate-related information and the blood oxygen level-related information and enables the signal processing circuit to enter a low power consumption mode.
